(19) United States
(12) Patent Application Publication
(10) Pub. No.: US 2013/0296571 A1
(43) Pub. Date: Nov. 7, 2013

(54) QUINOLINE DERIVATIVE COMPOUND, METHOD FOR PREPARING SAME, AND PHARMACEUTICAL COMPOSITION CONTAINING SAME

(71) Applicants: Miwon Son, Gyeonggi-do (KR); Tae Ho Lee, Gyeonggi-do (KR); Sang Zin Choi, Gyeonggi-do (KR); Nam Joon Baek, Seoul (KR); Kang Ro Lee, Gyeonggi-do (KR); Ki Hyun Kim, Gyeonggi-do (KR); Soon Hoe Kim, Gyeonggi-do (KR)

(72) Inventors: Miwon Son, Gyeonggi-do (KR); Tae Ho Lee, Gyeonggi-do (KR); Sang Zin Choi, Gyeonggi-do (KR); Nam Joon Baek, Seoul (KR); Kang Ro Lee, Gyeonggi-do (KR); Ki Hyun Kim, Gyeonggi-do (KR); Soon Hoe Kim, Gyeonggi-do (KR)

(73) Assignee: Dong-A ST Co., Ltd., Seoul (KR)

(21) Appl. No.: 13/989,407

(22) PCT Filed: Nov. 24, 2010
(86) PCT No.: PCT/KR2010/008349

§ 371 Date: Jul. 12, 2013

(51) Int. Cl. C07D 221/18

(52) U.S. Cl. CPC C07D 221/18
USPC 546/75

(57) ABSTRACT

The present invention relates to a novel quinoline derivative compound, an optical isomer thereof, a pharmaceutically acceptable salt thereof, and a hydrate or a solvate thereof. The novel quinoline derivative compound, the optical isomer thereof, the pharmaceutically acceptable salt thereof, and the hydrate or the solvate thereof accelerates gastrointestinal movement, and thus can effectively prevent or treat gastrointestinal motility disorders.

### TECHNICAL FIELD

The present invention relates to a quinoline derivative compound, an optical isomer thereof, a pharmaceutically acceptable salt thereof, or a hydrate or solvate thereof, a method for the preparation thereof, and a pharmaceutical composition comprising the same.

### BACKGROUND ART

Pathological mechanisms of functional gastrointestinal diseases such as gastrointestinal motility disorders act together, manifesting one or usually multiple symptoms. Depending on the symptoms, functional gastrointestinal diseases are classified into ulcer-like dyspepsia, dysmotility-like dyspepsia, reflux-like dyspepsia, nonspecific or unspecified functional dyspepsia.

Functional gastrointestinal disorders have been found in 25˜50% of the world population, with patients in need of therapeutic care accounting for about 5% of all the diagnosed cases. Functional gastrointestinal disorders are generated at greatly different rates from one region to another in the world, with relatively low incidence in the occident, for example, 22% for the U. K. and 19% for the U.S., whereas the incidence increases to 35-42% in Japan and to 62% in East Africa. Digestive tract diseases rank as the second most abundant among all diseases in South Korea. Particularly, the prevalence of gastric diseases is high, with functional gastrointestinal disorders diagnosed in 30˜40% of Korean population. As described above, since functional gastrointestinal diseases exhibit high morbidity and severe symptoms, leading to a deterioration in the quality of a patient's life and a need for frequent medical examination and treatment, thus presenting inconvenience to daily work schedule, an effective treatment is necessary.

Symptomatic causes of these functional gastrointestinal diseases usually remain unclear, although some of them have been identified. Such cryptogenic functional gastrointestinal diseases cannot be defined by histopathological and biochemical organic lesions, and are therefore explained in terms of functional symptoms. Various symptoms that manifest in functional gastrointestinal disorders may be treated mainly by promoting gastrointestinal motility.

Functional dyspepsia, which is a representative gastrointestinal motility disorder, is also diagnosed based on various dyspepsia symptoms without apparent organic lesions, and therefore therapy thereof is not simple, and most symptoms fluctuate between amelioration and deterioration, depending on various factors including diet and stress. These pathological mechanisms act together, manifesting one or usually multiple symptoms. For example, symptoms of functional dyspepsia include post-meal satiety, anorexia, sense of abdominal fullness, early satiety, belching, epigastric discomfort or pain, nausea, brash, vomiting, acid reflux, and heartburn, and functional dyspepsia still remain unknown in terms of pathophysiology (Panganamamula et. al., Functional (Nonulcer) Dyspepsia, Current Treatment Options in Gastroenterology, 5, pp. 153-160, 2002).

Representative among therapeutics for functional dyspepsia are domperidone, metoclopramide, levosulpiride, mosapride, itopride, and erythromycin, which are all gastrointestinal prokinetic agents. For the treatment of brash and ulcer, which are representative symptoms of functional dyspepsia, gastric acid suppressants and gastric antacids are used, but these drugs such as H2 antagonists, usually have temporary effects (Vincenzo Stanghellini et al., Delayed Gastric Emptying in Functional Dyspepsia, Current Treatment Options in Gastroenterology, 7, 259-264, 2004).

5-HT4 receptor agonists, a kind of gastroprokinetic agents which have recently been used as therapeutics for functional dyspepsia, were found to improve the symptoms without increasing a strain in the gastric fundus. Cisapride, one of these 5-HT4 receptor agonists, functions to promote gastric emptying, with a high statistic significance compared to other drugs. As to the duodenum or intragastric pressure wavelength (6> cm), however, cispride increases thresholds for the perception of gastric distension in healthy individuals as well as in patients, and may also exhibit adverse side effects to the heart that are potentially fatal.

Other conventional gastroprokinetic agents are far inferior to cisapride in therapeutic effect, and there is therefore a need to develop a therapeutic for functional dyspepsia or gastrointestinal motility disorders that exerts excellent therapeutic effects, without provoking side effects.

### DISCLOSURE

### Technical Problem

The present invention provides a novel quinoline derivative compound, an optical isomer thereof, a pharmaceutically acceptable salt thereof, and a hydrate or solvate thereof.

The present invention provides a method for preparing a novel quinoline derivative compound, an optical isomer thereof, a pharmaceutically acceptable salt thereof, or a hydrate or solvate thereof.

The present invention provides a pharmaceutical composition for the prevention or treatment of a gastrointestinal motility disorder, comprising a novel quinoline derivative compound, an optical isomer thereof, a pharmaceutically acceptable salt thereof, or a hydrate or solvate thereof.

The present invention provides the use of a composition comprising a novel quinoline derivative compound, an optical isomer thereof, a pharmaceutically acceptable salt thereof, or a hydrate or solvate thereof in the prevention or treatment of a gastrointestinal motility disorder.

The present invention provides a method for preventing or treating a gastrointestinal motility disorder using a quinoline derivative compound, an optical isomer thereof, a pharmaceutically acceptable salt thereof, or a hydrate or solvate thereof.

### Technical Solution

The present invention provides a quinoline derivative compound represented by the following Chemical Formula 1, an optical isomer thereof, a pharmaceutically acceptable salt thereof, and a hydrate or solvate thereof:

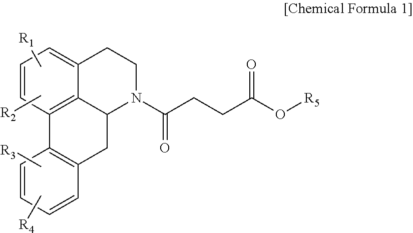

wherein for Chemical Formula 1:
R₁ to R₄ are independently —H or methoxy; and
R₅ is linear or branched alkyl of C₁ to C₄.

The compound represented by Chemical Formula 1 may include an asymmetric carbon atom. In this context, the present invention may comprise not only the compound represented by Chemical Formula 1, but also the optical isomer thereof.

In accordance with the present invention, the quinoline derivative compound represented by Chemical Formula 1 may be a compound represented by the following Chemical Formula 2:

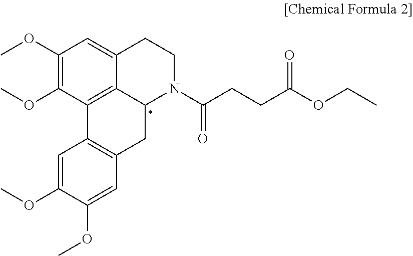

wherein represents an asymmetric carbon atom.

Thus, the present invention includes the compound represented by Chemical Formula 2, an optical isomer thereof, a pharmaceutically acceptable salt thereof, a hydrate thereof, and a solvate thereof.

The present invention includes (S)-4-oxo-4-(1,2,9,10-tetramethoxy-4,5,6a,7-tetrahydro-dibenzo[de,g]quinoline-6-yl)-butyric acid ethyl ester, an optical isomer thereof, a pharmaceutically acceptable salt thereof, and a hydrate or solvate thereof.

As used herein, the term “pharmaceutically acceptable salt” refers to any of typical salts employed in the pharmaceutical field. Examples of the salts include inorganic ion salts such as salts of calcium, potassium, sodium and magnesium, inorganic acid salts such as salts of hydrochloric acid, nitric acid, phosphoric acid, bromic acid, iodic acid, perchloric acid, tartaric acid, and sulfuric acid, organic acid salts such as salts of acetic acid, trifluoroacetic acid, citric acid, maleic acid, succinic acid, oxalic acid, benzoic acid, tartaric acid, fumaric acid, mandelic acid, propionic acid, citric acid, lactic acid, glycolic acid, gluconic acid, galaturonic acid, glutamic acid, glutaric acid, glucuronic acid, aspartic acid, ascorbic acid, carbonic acid, vanillic acid, hydroiodic acid, mandelic acid, mucic acid, nitric acid, pamoic acid, pantothenic acid, succinic acid, and tartaric acid, and sulfonic acid salts such as salts of methane sulfonic acid, ethane sulfonic acid, benzene sulfonic acid, p-toluene sulfonic acid, camphoric acid, and naphthalene sulfonic acid, amino acid salts such as salts of glycine, arginine, and lysine, and amine salts such as salts of trimethylamine, triethylamine, ammonia, pyridine, and picoline, but are not limited thereto. For example, pharmaceutically acceptable salts are hydrochloric acid as the inorganic acid salts and methane sulfonic acid as the organic acid salts, respectively.

A hydrate of the compound of Chemical Formula 1 or 2, the above-suggested compounds, or optical isomers or the pharmaceutically acceptable salts thereof may include a stoichiometric or non-stoichiometric amount of water bound by non-covalent intermolecular forces. The hydrate may include at least one equivalent of water, for example, one to five equivalents of water. It may be prepared by crystallizing the compounds, optical isomers thereof, or pharmaceutically acceptable salts thereof in water or an aqueous solvent.

A solvate of the compounds of Chemical Formula 1 or 2, the above-suggested compounds, or the optical isomers or the pharmaceutically acceptable salts thereof may comprise a stoichiometric or non-stoichiometric amount of a solvent bound by non-covalent intermolecular forces. Preferred is a solvent which is non-volatile, non-toxic, and suitable for administration to humans. Ethanol, methanol, propanol, and methylene chloride may be exemplary.

The compounds represented by Chemical Formula 1 or 2, the above-suggested compounds, the optical isomers, the pharmaceutically acceptable salts, or the hydrates or solvates thereof are prophylactic or therapeutic for gastrointestinal motility disorders. Having affinity for dopamine-D2 receptors, serotonin-1A receptors, and serotonin-1B receptors, the compounds of the present invention, that is, the compounds represented by Chemical Formula 1 or 2, the above-suggested compounds, the optical isomers, the pharmaceutically acceptable salts or the hydrate or solvates thereof can be used for the prevention or treatment of various symptoms of gastrointestinal motility disorders through interaction with the receptors.

The present invention provides a method for preparing a compound represented by the following Chemical Formula 1, an optical isomer thereof, a pharmaceutically acceptable salt thereof, and a hydrate or solvate thereof, comprising reacting a compound represented by the following Chemical Formula 3 or an optical isomer thereof with a compound represented by the following Chemical Formula 4 or an optical isomer thereof in the presence of a base:

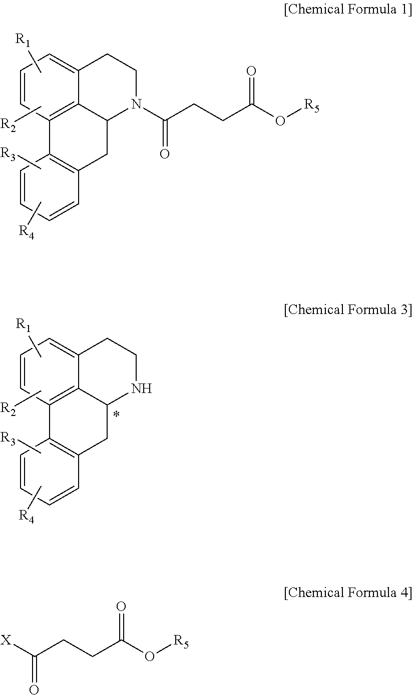

wherein for Chemical Formulas 1 to 4:
R₁ to R₄ are independently —H or methoxy;
R₅ is linear or branched alkyl of C₁ to C₄;
X is Cl, Br or I; and
* represents an asymmetric carbon atom.

The compound represented by Chemical Formula 3 or an optical isomer thereof may be prepared using a typical method known in the art, or may be commercially available. For example, the compound may be obtained from an extract of Corydalis tuber using a well-known method (Tae Ho Lee et al., Biol. Pharm. Bull. 33(6) 958-962 (2010), “Effects of Corydaline from Corydalis Tuber on Gastric Motor Function in an Animal Model”, Ki Hyun Kim et al., Planta Med. 2010 May 27, “New Cytotoxic Tetrahydroprotoberberine-Aporphine Dimeric and Aporphine Alkaloids from Corydalis Turtschaminovii”).

In the method of the present invention, 1,2,9,10-tetramethoxy-5,6,6a,7-tetrahydro-4H-dibenzo[de,g]quinoline-((+)-norglausine), and ethyl 4-chloro-4-oxobutanoate may be used as the compounds represented by Chemical Formulas 3 and 4, respectively.

The preparation method of the present invention may include the reaction illustrated in the following Reaction Scheme 1:

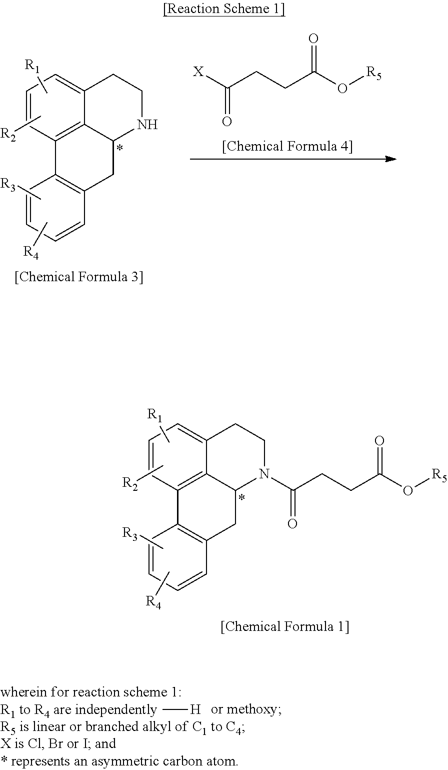

In Reaction Scheme 1, R₁ to R₄ may be methoxy, R₅ may be ethyl, and X may be Cl.

As can be understood from Reaction Scheme 1 according to the method of present invention, the optical purity of Chemical Formula 1 may be determined by the optical purity of the compound represented by Chemical Formula 3. Hence, given optical purity, the compound of Chemical Formula 3 gives a predetermined optical structure to the compound of Chemical Formula 1 according to the method of the present invention. For example, if the compound of Chemical Formula 3 has an (S)-configuration, the compound of Chemical Formula 1 is obtained as an(S) enantiomer.

On the other hand, when the compound of Chemical Formula 3 is a racemate, the compound of Chemical Formula 1 may be obtained in a racemic mixture. In this case, the mixture can be separated into desired enantiomers using a well-known method.

The reaction illustrated in Reaction Scheme 1 may be carried out in an organic solvent. Particular limitations are not burdened on the kind of the organic solvents, and examples of the organic solvent used in the reaction may include methanol, ethanol, propanol, dimethylsulfoxide (DMSO), tetrahydrofuran (THF), acetonitrile (ACN), dimethylformamide (DMF), N-methylpyrrolidinone (NMP), dimethylacetamide (DMA), or mixtures thereof.

In the presence of the base, the reaction of Reaction Scheme 1 may be carried out. The base useful in the reaction may be obtained from various sources, and examples of the base may include triethylamine, N,N-diisopropylethylamine, N-methylporphyrin, N-methylpiperidine, 4-dimethylaminopyridine, N,N-dimethylaniline, 2,6-rutidine, tricalcium phosphate, potassium carbonate, pyridine, or a mixture thereof, and the base may preferably include trimethylamine.

In accordance with a further aspect thereof, the present invention provides a pharmaceutical composition for the prevention or treatment of a gastrointestinal motility disorder, comprising a novel quinoline derivative compound represented by the following Chemical Formula 1, an optical isomer thereof, a pharmaceutically acceptable salt thereof, or a hydrate or solvate thereof:

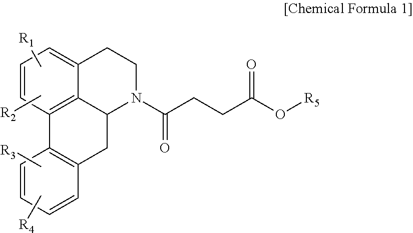

wherein for Chemical Formula 1:
R₁ to R₄ are independently —H or methoxy; and
R₅ is linear or branched alkyl of C₁ to C₄.

The pharmaceutical composition of the present invention may include a compound represented by the following Chemical Formula 2, an optical isomer thereof, a pharmaceutically acceptable salt thereof, or a hydrate or solvate thereof:

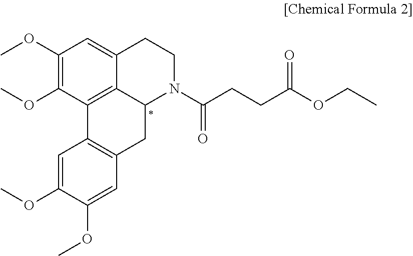

wherein represents an asymmetrical carbon atom.

The pharmaceutical composition of the present invention may preferably include (S)-4-oxo-4-(1,2,9,10-tetramethoxy-4,5,6a,7-tetrahydro-dibenzo[de,g]quinoline-6-yl)-butyric acid ethyl ester, an optimal isomer thereof, a pharmaceutically acceptable salt thereof, or a hydrate or solvate thereof.

In the pharmaceutical composition, the pharmaceutically acceptable salt, the hydrate, and the solvate may be the same as defined above.

The pharmaceutical composition of the present invention may be applicable to the prevention or treatment of functional gastrointestinal disorders, such as gastrointestinal motility disorder.

Examples of the gastrointestinal motility disorder may include functional dyspepsia resulting in symptoms such as early satiety, pain, epigastric distress, a false sense of satiety, heartburn, nausea and vomiting, etc., ulcerative dyspepsia, non-ulcerative dyspepsia, a gastroesophageal reflux disease, reflux esophagitis, gastric atony, intestinal pseudo-obstruction, gastroparesis, constipation, irritable bowel syndrome, general hypersensitive colitis, hypersensitive colitis accompanied by constipation, hypersensitive colitis accompanied by diarrhea, a diabetic gastrointestinal motility disorder, a chemotherapy-induced gastrointestinal motility disorder, a visceral obstruction due to gastrointestinal dysmotility, a gastrointestinal motility disorder due to myotonic dystrophy, and noncardiac chest pain.

Of the gastrointestinal motility disorders, functional dyspepsia does not result in a pathological or biochemical organic lesion, but rather it gives rise to functional symptoms with manifestation of continuous epigastric distress or pain. Medically, this means various symptoms associated with continuous and repetitive discomfort or pain confined to the epigastric part. Specifically, functional dyspepsia may include all the symptoms relating to the digestive system including postprandial fullness, anorexia, abdominal distention, early satiety, belching, epigastric distress or pain, brash, nausea, vomiting, gastric reflux and heartburn, etc.

It is known that about 30% of patients with functional dyspepsia exhibit gastrointestinal dysmotility such as delaying of gastric emptying time taken to transfer foods to the small intestine through the pylorus of the stomach. In addition, early satiety takes place even with a small amount of food intake, due to a decline of gastric accommodation which maintains intragastric pressure by appropriate expansion of the stomach in response to intake of food.

The pharmaceutical composition of the present invention can improve gastric emptying of foods, gastric accommodation, and gastrointestinal transit and activate gastric motility. Therefore, the pharmaceutical composition of the present invention may be effectively prophylactic or therapeutic for functional dyspepsia or various symptoms of gastrointestinal motility disorder.

In addition, the pharmaceutical composition of the present invention may be capable of improving gastric emptying capacity, gastric accommodation and gastrointestinal transit, without causing adverse side effects such as adverse effects on the heart, thereby contributing to an improvement in gastrointestinal motility.

With high affinity for dopamine-D2, serotonin-1A, and serotonin-1B receptors, the pharmaceutical composition of the present invention can exert preventive or therapeutic effects on various symptoms and disorders exhibited in gastrointestinal motility disorders through interaction with the receptors.

The pharmaceutical composition of the present invention has high affinity for the dopamine-D2 receptor. The dopamine-D2 receptor, found in the wall of digestive ducts of various mammals, is known to inhibit gastrointestinal motility. Domperidone and metocloprimide, both selective antagonists for the inhibitory receptor dopamine-D2, promotes gastrointestinal motility. Also, as dopamine-D2 receptor antagonists that inhibit vomiting, levosulpiride, clebopride, bromopride, etc. are used as antiemetics with prompting gastroprokinetic activity in some countries (P. Moayyedi, S. Soo, J. Deeks, B. Delaney, M. Innes and D. Forman, Pharmacological interventions for non-ulcer dyspepsia, Cochrane Database SystRev18, 2006; G. Karamanolis and J. Tack, Promotility medications-now and in the future, DigDis24, 2006 pp. 297-307). Thus, the pharmaceutical composition of the present invention acts as a dopamine-D2 receptor antagonist with high affinity for the dopamine-D2 receptor, so that it can promote gastrointestinal motility.

The pharmaceutical composition of the present invention shows high affinity for the serotonin-1A receptor. This receptor functions to control gastric accommodation, and buspirone, an agonist for the serotonin-1A receptor, increases gastric accommodation in a dose-dependent manner in human test. In addition, R-137696, a serotonin-1A agonist under development, was found to relax the proximal stomach in a dose-dependent manner in a human test (G. E. Boeckxstaens, G. N. Tytgat, E. Wajs, L. van Nueten, F. de Ridder, A. Meulemans and J. Tack, The influence of the novel 5-HT 1A agonist R137696 on the proximal stomach function in healthy volunteers, Neurogastroenterol Motil 182006, pp. 919-926). Therefore, the pharmaceutical composition of the present invention, which exhibits affinity for the serotonin-1A receptor, can improve gastric accommodation.

The pharmaceutical composition of the present invention also shows High affinity for the serotonin-1B receptor. Sumatriptan, a serotonin-1B receptor agonist, exerts a relaxation effect on the stomach and reduces meal-induced satiety in functional dyspepsia patients, thereby exhibiting a therapeutic activity for impaired gastric accommodation (Tack, H. Piessevaux, B. Coulie, P. Caenepeel and J. Janssens, Role of impaired gastric accommodation to a meal in functional dyspepsia, Gastroenterology 115 (1998), pp. 1346-1352; J. Tack, P. VandenBerghe, B. Coulie and J. Janssens, Sumatriptan is anagonist at 5-HT receptors on myenteric neurones in the guinea-pig gastric antrum, Neurogastroenterol Motil (2007), pp. 39-46). Therefore, the pharmaceutical composition of the present invention can be used as a prophylactic or therapeutic agent for impaired gastric accommodation because it exhibits stomach relaxation and reduction in meal-induced satiety.

The pharmaceutical composition of the present invention may include at least one of the compounds represented by Chemical Formula 1 or 2, the above-suggested compound, optical isomers thereof, pharmaceutically acceptable salts thereof, hydrates thereof, and solvates thereof. For example, the pharmaceutical composition of the present invention may include one or two ingredients of the compounds represented by Chemical Formula 1 or 2, the above-suggested compound, optical isomers thereof, pharmaceutically acceptable salts thereof, hydrates thereof, and solvates thereof.

In addition to the compounds represented by Chemical Formula 1 or 2, the above-suggested compound, optical isomers thereof, pharmaceutically acceptable salts thereof, hydrates thereof, and solvates thereof, the pharmaceutical composition of the present invention may include another ingredient effective for the prevention or treatment of gastrointestinal motility disorders. For example, the pharmaceutical composition may further include domperidone, metoclopramide, levosulpiride, mosapride, itopride, or erythromycin.

The pharmaceutical composition of the present invention may further a pharmaceutically active ingredient effective for the therapy of diseases other than dyspepsia or gastrointestinal motility disorders.

To effect the prevention or treatment of gastrointestinal motility disorders, the pharmaceutical composition of the present invention may be used alone or in conjunction with a surgical operation, hormonal therapy, a drug regimen, or an agent for biological modulation.

For proper administration, the pharmaceutical composition of the present invention may include at least one pharmaceutically acceptable carrier in addition to the compound of Chemical Formula 1 or 2, an optical isomer, a pharmaceutically acceptable salt, or a hydrate or solvate thereof. The pharmaceutically acceptable carrier may include saline, sterile water, Ringer's solution, buffered saline, dextrose solution, maltodextrin solution, glycerol, ethanol, or a combination thereof. If necessary, the pharmaceutical composition may further include a typical additive such as an antioxidant, buffer, a bacteriostat, etc.

According to purpose, the pharmaceutical composition of the present invention may be administered orally or parenterally (e.g., intravenously, subcutaneously, intradermally, or topically). The therapeutically effective amount of the active ingredient may vary depending on various factors including a patient's age, weight, gender, health condition, and diet, the time of administration, the route of administration, a period or an interval of the administration, the rate of excretion, constitutional disposition, the property of a formulation to be administered, and the severity of disease, etc. The compound of Chemical Formula 1, an optical isomer thereof, a pharmaceutically acceptable salt thereof, or a hydrate or solvate thereof may be administered in an amount of from 0.01 to 100 mg/kg, and preferably in an amount of from 0.1 to 30 mg/kg, once a day, or may be divided till triple doses a day.

For administration, the pharmaceutical composition of the present invention may be formulated into various dosage forms. The pharmaceutical composition may be formulated together with a pharmaceutically acceptable carrier into various pharmaceutical dosage forms. The carrier may be a non-toxic, inert formulation auxiliary agent that may be in any phase, such as a solid, quasi-solid, or liquid phase, and pharmaceutically acceptable. For example, a filler, a thickener, a binder, a wetting agent, a disintegrant, a dispersant, a surfactant, or a diluent may be employed.

The pharmaceutical composition of the present invention may be prepared into unit dosage forms. In the unit dosage forms, the compound of Chemical Formula 1 or 2, an optical isomer, a pharmaceutically acceptable salt, a hydrate or a solvate thereof may be present in an amount corresponding to a fraction or a multiple of the daily dose thereof. For example, the unit dosage form may contain the active ingredient in an amount 1, 2, 3 or 4, or ½, ⅓ or ¼ times as much as a necessary daily dose thereof. Preferably, the amount of the active ingredient in a unit dosage form is a single dose which typically corresponds to daily dose or, ½, ⅓ or ¼ of the daily dose.

The pharmaceutical composition of the present invention may be formulated into a tablet, a coated tablet, a capsule, a pill, a granule, a suppository, a liquid, a suspension, an emulsion, a paste, an ointment, a gel, a cream, a lotion, a powder, or a spray. For oral administration, for example, the pharmaceutical composition of the present invention may be formulated into a solid agent, such as a tablet, a pill, a powder, a granule or a capsule, or a liquid agent such a suspension, an internal use liquid, an emulsion or a syrup. Alternatively, the pharmaceutical composition of the present invention may be administered via a parenteral route. For this, the pharmaceutical composition of the present invention may be in the form of an injection, a suspension, an emulsion, a lyophilizate, or a suppository. For example, the compound of Chemical Formula 1, or an optical isomer, a pharmaceutically acceptable salt thereof, a hydrate or a solvate thereof may be formulated together with at least one excipient into a microcapsule.

When the pharmaceutical composition of the present invention is formulated into a solid preparation such as a tablet, a coated tablet, a capsule, a pill or a granule, the active ingredient may be combined with (a) a filler and a thickener, such as starch, lactose, sucrose, glucose, mannitol, or silicate, (b) a binder, such as carboxymethyl cellulose, alginate, gelatin, polyethylene glycol, microcrystalline cellulose, highly dispersible silica, natural gum, synthetic gum, povidone, copovidone, polyvinyl pyrrolidone, or gelatin, (c) a moisture absorbent such as glycerol, (d) a disintegrant such as agar, calcium carbonate, or sodium carbonate, (e) a dissolution retardant such as paraffin, (f) an absorption accelerator such as a quaternary ammonium compound, (g) a wetting agent such as cetyl alcohol, or glycerol monostearate, (h) an absorber such as kaolin or bentonite, or (i) a lubricant such as talc, calcium stearate, magnesium stearate, talc or a solid polyethylene glycol, or a mixture of (a) to (i).

For formulating the pharmaceutical composition of the present invention into a liquid agent for oral administration, such as a suspension, an internal use liquid, or a syrup, various additives including a diluent such as water, liquid paraffin, etc., a wetting agent, a sweetener, anodorant, a preservative, antiseptic and a colorant may be added as necessary. For example, peppermint oil, eucalyptus oil or a sweetener such as saccharin may be added to the pharmaceutical composition of the present invention.

When the pharmaceutical composition of the present invention is formulated into a suppository, a water-soluble or insoluble excipient, for instance, a lipid such as polyethylene glycol, cacao lipid, etc., a high ester (e.g., C14-alcohol with C16-fatty acid), Witepsol, macrogol, Tween 61, laurin butter, glycerol gelatin, or a combination thereof may be employed.

Also, the pharmaceutical composition of the present invention may take the form of ointments, pastes, creams or gels. In this regard, animal or vegetable lipids, wax paraffins, starch, tragacanth, cellulose derivatives, polyethylene glycol, silicon, bentonite, silicic acid, talc, zinc oxide or a combination thereof may be used.

For a powder or spray, the pharmaceutical composition of the present invention may be formulated in combination with lactose, talc, silicic acid, aluminum oxide, calcium silicate, polyamide powder, or a mixture thereof. A spray formulation may further comprise a typical propellant such as chlorofluorohydrocarbon. PEG-4000 and glycerin may be typically needed to obtain an inhalation spray.

In the case of liquid or emulsion formulations intended for the parenteral administration of the pharmaceutical composition of the present invention, their formulations may be obtained with a solvent, a dissolving agent, or an emulsifier. For example, water, ethyl alcohol, isopropyl alcohol, ethyl carbonate, ethyl acetate, benzyl alcohol, benzyl benzoate, propylene glycol, 1,3-butylene glycol, dimethyl formamide, an oil such as cotton seed oil, peanut oil, corn seed oil, olive oil, castor oil, or sesame oil, glycerol, glycerol formalcohol, tetrahydrofurfuryl alcohol, polyethylene glycol, a fatty acid of sorbitan, or a combination thereof may be used to formulate the composition of the present invention into a liquid or an emulsion.

The liquid or emulsion for parenteral administration may be in a sterile, and blood-isotonic state.

For use in formulating the pharmaceutical composition into a suspension, a liquid diluent such as water, ethyl alcohol, propylene glycol, or polyethylene glycol, ethoxylated isostearyl alcohol, polyoxyethylene sorbitol and sorbitan ester, microcrystalline cellulose, aluminum metahydroxide, bentonite, agar, tragacanth, an injectable ester such as ethyl oleate, and a mixture thereof may be suitable.

In addition, the pharmaceutical composition of the present invention may be formulated into a sustained or immediate release form with an excipient, a diluent, a dispersant, a surfactant, a binder, a lubricant or an additive. For example, the pharmaceutical of the present invention may be formulated into the sustained or immediate release form in order to release the compound of Chemical Formula 1 or 2, or an optical isomer, a pharmaceutically acceptable salt, a hydrate or a solvate thereof in the predetermined organ of the body.

The pharmaceutical composition of the present invention may be formulated into the sustained release form using a sustained release agent such as an enteric coating agent, a water-insoluble polymer a hydrophobic compound, a polymeric material such as a hydrophilic polymer, or an embedding agent such as wax. For example, the pharmaceutical composition takes a form of tablets, capsules, pills or granules, a coating film may be applied to them to achieve a sustained release.

No particular limitations are imparted to the amount of the additives used in preparing a formulation of the pharmaceutical composition of the present invention, such as carriers, fillers, thickeners, binders, wetting agents, disintegrants, dispersants, surfactants, or diluents. The amount of additives may be suitably adjusted within the range typically used in typical formulations.

Using a suitable method known in the art, the pharmaceutical composition of the present invention may be formulated depending on the disease to be treated or the ingredient to be used. For example, the compound of Chemical Formula 1 or 2, the above-suggested compound, or an optical isomer or a pharmaceutically acceptable salt thereof may be mixed with an excipient to be formulated into a desired preparation.

In the pharmaceutical composition of the present invention, the active ingredient, such as the compound of Chemical Formulas 1 or 2, the above-suggested compound, or an optical isomer, a pharmaceutically acceptable salt, a hydrate or a solvate thereof, may be employed in an amount of from about 0.1 to about 99.5 wt %, and preferably in an amount of from about 0.5 wt % to about 95 wt %.

Also, the present invention provides the use of the pharmaceutical composition including the compound of Chemical Formula 1 or 2, or an optical isomer, a pharmaceutically acceptable salt, a hydrate or a solvate thereof in the treatment of a gastrointestinal motility disorder.

The present invention provides a method for preventing or treating dyspepsia or a gastrointestinal motility disorder, using the compound of Chemical Formula 1 or 2, or an optical isomer, a pharmaceutically acceptable salt, a hydrate or a solvate thereof. The pharmaceutical composition may be administered into the subject including a human to prevent or treat dyspepsia or a gastrointestinal motility disorder.

### Advantageous Effects

As described hitherto, the novel quinoline derivative compounds, optical isomers thereof, pharmaceutically acceptable salts thereof, and hydrates or solvates thereof can activate gastrointestinal motility. Compositions including the novel quinoline derivative compounds, optical isomers thereof, pharmaceutically acceptable salts thereof, or and hydrates or solvates thereof can be effectively applied to the prophylaxis and therapy of functional dyspepsia or various symptoms of gastrointestinal motility disorders.

### MODE FOR INVENTION

A better understanding of the present invention may be obtained through the following examples which are set forth to illustrate, but are not to be construed as limiting the present invention.

Unless stated otherwise, reagents and solvents used in the following Example section were purchased from Sigma-Aldrich. (+)-Norglausine was obtained from an extract of Corydalis tuber using a well-known method (Tae Ho Lee et al., Biol. Pharm. Bull. 33(6) 958-962 (2010), “Effects of Corydaline from Corydalis Tuber on Gastric Motor Function in an Animal Model,” Ki Hyun Kim et al., Planta Med. 2010 May 27, “New Cytotoxic Tetrahydroprotoberberine-Aporphine Dimeric and Aporphine Alkaloides from Corydalis Turtschaminovii”).

¹H-NMR data were measured using a UNITY INOVA 500 NMR spectrometer, Varian. IR data were measured using an IFS-66/S FT-IR spectrometer, Bruker, UV data were measured using an UV-1601 UV-visible spectrophotometer, Shimadzu, and CD data were measured using a J-715 spectropolarimeter, JASCO. For mass data, a JMS700 mass spectrometer, JEOL, was employed.

### Example 1

### Preparation of (S)-4-oxo-4-(1,2,9,10-tetramethoxy-4,5,6a,7-tetrahydro-dibenzo[de,g]quinoline-6-yl)-butyric acid ethyl ester

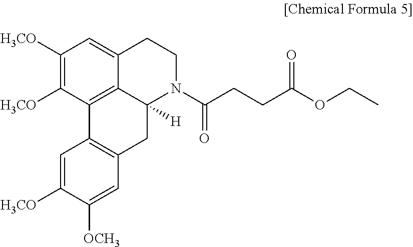 [SMILES: CCOC(=O)CCC(=O)N1CCc2cc(OC)c(OC)c3c2[C@@H]1Cc1cc(OC)c(OC)cc1-3]

In 10 mL of benzene were dissolved 200 mg of 1,2,9,10-tetramethoxy-5,6,6a,7-tetrahydro-4H-dibenzo[de,g]quinoline ((+)-norglausine) and 0.24 ml of triethylamine, followed by dropwise addition of a solution of 0.17 mL of ethyl 4-chloro-4-oxobutanoate (Sigma-Aldrich) dissolved in 10 mL of anhydrous tetrahydrofuran and then by stirring for 2 hrs under reflux. The resulting reaction mixture was cooled to room temperature and filtered, and the filtrate was concentrated at a reduced pressure. After extraction using with water and ethyl acetate, the extract was dried over anhydrous magnesium sulfate. Thereafter, the solvent was removed by distillation under reduced pressure. Then 220 mg of the title compound, represented by Chemical Formula 5 was obtained by silica gel column chromatography.

IR (KBr): νₘₐₓ=3388, 2945, 1667, 1517, 1461, 1254, 1030, 676 cm⁻¹;

UVλₘₐₓ(MeOH)nm(log ε): 216 (3.75), 232 (2.75), 245 (3.12), 279 (4.42), 299 (4.80), 311 (4.27);

CD (MeOH) λₘₐₓ(Δε) 216 (+22.4), 238 (+12.4), 243 (+8.2), 263 (+4.3), 279 (−12.8), 299 (+15.7)nm;

FAB-MS: m/z=492[M+Na]⁺;

HR-FAB-MS: m/z=492.1995[M+Na]⁺ (calcd. for C₂₆H₃₁NNaO₇: 492.1998);

¹H-NMR (CD₃OD, 500 MHz): 1.23 (t, 3H), 2.64 (m, 2H), 2.65 (m, 2H), 2.74 (m, 1H), 2.80 (t, 1H), 3.06 (dd, 1H), 3.23 (m, 1H), 3.28 (m, 1H), 3.64 (d, 1H), 3.64 (s, 3H), 3.89 (s, 3H), 3.90 (s, 3H), 3.93 (s, 3H), 6.60 (s, 1H), 4.11 (q, 2H), 6.60 (s, 1H), 6.79 (s, 1H), 8.09 (s, 1H);

¹³C-NMR (CD₃OD, 125 MHz): 14.3, 27.5, 29.5, 29.5, 32.2, 53.2, 56.1 (x2), 56.2 (x2), 60.5, 60.9, 110.3, 111.1, 111.8, 121.5, 123.9, 127.1, 127.5, 129.5, 145.3, 148.2, 148.8, 153.5, 172.7, 176.4

Although the following provides a more detailed explanation of the present invention by indicating Experimental Examples, the present invention is not limited thereto.

### Experimental Example 1

### Assay for Affinity for Dopamine Receptor

Affinity of the compounds of the present invention for the dopamine-D2 receptor, which is found in the wall of the gastric tract of mammals and inhibits gastric motility, was determined by measuring competitive inhibition against the binding to the receptor of a radio-labeled ligand which is known for its affinity for the receptor. In this regard, the experiment was performed according to the method described in [Heys G et al. Mol. Endocrinol. 6:920, 1992] and [Grandy D K et al. Proc Natl Acad Sci USA. 86:9762, 1989].

In tris-HCl buffer (50 nM Tris-HCl, pH 7.4, 120 mM NaCl, 5 mM KCl, 2 mM CaCl₂, 1 mM MgCl₂) 150 μg of Chinese hamster ovary (CHO) cells transfected with a human dopamine D2, gene were incubated at 25° C. for 120 min with 0.16 nM of tritium Spiperone (³[H] Spiperon [NEN, 250 μCi]) and 10 μM of the compound of interest. After completion of the incubation, the incubation mixture was filtered through Whatman GF/B filter (unifilter-96 Lot: 6005177, PerkinElmer) to separate ligand-bound receptors. These bound receptors were washed three times with a Tris-HCl buffer. Then, 10 mL of a scintillation cocktail (Lot: 03999, Fluka) was added and reacted for 16 hrs or longer to measure radiation activity using a beta-counter (Packcard) in order to calculate the quantity of tritium spiperone bound to the receptor. Meanwhile, 10 μM of Haloperidol (H1512, Sigma) was incubated in the presence of 0.16 nM tritium Spiperone to determine a nonspecific binding value. The experiment was independently carried out twice, and receptor affinity was calculated using the following Equation 1. A mean value of the measurements is given in Table 1, below.

Receptor Affinity(%)={(Total CPM−Nonspecific Binding CPM−CPM of tritium Spiperone remaining after addition of interest compound of the present invention)/(Total CPM−Nonspecific Binding CPM)}×100(CPM:counter per minute) Equation 1

TABLE 1

| Example | Affinity (%) |
|---|---|
| Compound of Example | 46 |

As can be seen in Table 1, the compounds of the present invention at a concentration of 10 μM exhibited high affinity for the dopamine-D2 receptor. It is understood from the data that the compounds of the present invention function as a dopamine-D2 antagonist like domperidone, metochlopramide, or itopride, promoting gastrointestinal motility.

### Experimental Example 2

### Assay for Affinity for Serotonin-1A Receptor

Compounds of the present invention were examined for affinity for the serotonin-1A receptor which can increase gastric accommodation in response to an agonist.

In tris-HCl buffer (50 mM Tris-HCl, pH 7.4, 120 mM NaCl, 5 mM KCl, 2 mM CaCl₂, 1 mM MgCl₂), 150 μg of Chinese hamster ovary (CHO) cells transfected with a human dopamine 5-HT1A receptor were incubated at 37° C. for 1.5 hrs with 1.5 nM of ³[H] 8-OH-DPAT and 10 μM of the compound of interest of Example. After completion of the incubation, the incubation mixture was filtered through Whatman GF/B filter (unifilter-96, Lot: 6005177, PerkinElmer) to separate ligand-bound receptors. These bound receptors were washed three times with 5 mL of a Tris-HCl buffer. Then, 10 mL of a scintillation cocktail (Lot: 03999, Fluka) was added, and reacted for 16 hrs or longer with 10 mL of a scintillation cocktail (Lot: 03999, Fluka) to measure radiation activity using a beta-counter (Packcard) in order to calculate the quantity of ³[H] 8-OH-DPAT bound to the receptor.

Meanwhile, 10 μM of metergoline was also incubated in the presence of 1.5 nM of ³[H] 8-OH-DPAT to determine a nonspecific binding value. The experiment was independently carried out twice, and receptor affinity was calculated using the following Equation 2. A mean value of the measurements is given in Table 2, below.

Receptor Affinity(%)={(Total CPM−Nonspecific Binding CPM−CPM of ³[H]8-OH-DPAT remaining after addition of Interest compound of the present invention)/(Total CPM−Nonspecific Binding CPM)}×100(CPM:counter per minute) [Equation 2]

TABLE 2

| Example | Affinity (%) |
|---|---|
| Compound of Example | 41 |

As can be seen in Table 2, the compounds of the present invention at a concentration of 10 μM exhibited a certain affinity for the serotonin 1-A receptor. Thus, it is understood from the data that the compounds of the present invention can bind to the serotonin 1-A to improve gastric accommodation.

### Experimental Example 3

### Assay for Affinity for Serotonin-1B Receptor

Compounds of the present invention were examined for affinity for the serotonin-1B receptor which can exert a relaxation effect on the stomach and relieve impaired gastric accommodation of functional dyspepsia patients in response to an agonist.

In a Tris-HCl buffer (50 mM Tris-HCl, pH 7.4, 120 mM NaCl, 5 mM KCl, 2 mM CaCl₂, 1 mM MgCl₂), 4 mg of cell membranes of the cerebral cortex excised from male Winstar rats, each weighing 175±25 g, was incubated at 37° C. for 1.5 hrs with 10 μM of ¹²⁵[I] cyanopindolol (PerkinElmer, 100 μCi) and 10 μM of the compound of interest of the present invention per well. After completion of the incubation, the incubation mixture was filtered through Whatman GF/B filter (unifilter-96, Lot: 6005177, PerkinElmer) to separate ligand-bound receptors. These bound receptors were washed three times with 5 mL of a Tris-HCl buffer. Then, 10 mL of a scintillation cocktail (Lot: 03999, Fluka) was added, and reacted for 16 hrs or to measure a radiation activity using a beta-counter (Packcard) in order to calculate the quantity of ¹²⁵[I] cyanopindolol bound to the receptor.

Meanwhile, 10 μM of serotonin (H9523, Sigma) was incubated in the presence of 10 μM of ¹²⁵[I] cyanopindolol to determine a nonspecific binding value. The experiment was independently carried out twice, and receptor affinity was calculated using the following Equation 3. A mean value of the measurements is given in Table 3, below.

Receptor Affinity(%)={(Total CPM−Nonspecific Binding CPM−CPM of ¹²⁵[I]cyanopindolol remaining after addition of Interest compound of the present invention)/(Total CPM−Nonspecific Binding CPM)}×100(CPM:counter per minute) [Equation 3]

TABLE 3

| Example | Affinity (%) |
|---|---|
| Compound of Example | 92 |

As can be seen in Table 3, the compounds of the present invention at a concentration of 10 μM exhibited a high affinity for the serotonin 1-B receptor. Thus, it is understood from the data that the compounds of the present invention can bind to the serotonin 1-B to exert a relaxation effect on the stomach and relaxation effect on impaired gastric accommodation.

### Experimental Example 4

### Gastric Emptying Effect on Normal Rat

Gastric emptying activity of the compounds of the present invention was examined in the following experiment.

Sprague-Dawley rats with an age of 6 weeks were divided into groups according to weight, and acclimated for one week at a temperature of 22˜24° C. and a humidity of 60-80% while a standard diet and water were provided. Thereafter, they were starved for 24 hrs before an experiment while water was provided. From 3 hrs before the experiment, no more water was provided. A 3% hydroxy propyl methyl cellulose solution containing the compound of Example in an amount of 10 μg/kg was orally administered at a dose of 1 mL to each rat.

After 45 min, a semi-solid test meal (0.05% phenol red solution containing 1.5% MC), prepared by mashing feeds in water, was orally administered in an amount of 2 mL to each rat. After 35 min, the SD rats were killed, and the stomach was excised, and weighed carefully lest the gut content was leaked. Subsequently, the stomach was washed with distilled water to remove the gut content, dried, and weighed again, in order to calculate the weight of the semi-solid test meal left in the gut. Gastric emptying rate was calculated according to the following Equation 4, and the results are summarized in Table 4, below. In Table 4, the control was orally administered with 1 mL of 3% hydroxymethyl cellulose.

Gastric Emptying(%)=[1−(weight of semi-solid test meal left in gut/weight of semi-solid test meal at 0-time)]×100 [Equation 4]

(weight of semi-solid test meal at 0-time means weight of the semi-solid test meal left in the rat killed immediately after the administration of the semi-solid test meal)

TABLE 4

| Example | Dose (μ/kg) | Gastric Emptying (%) |
|---|---|---|
| Example | 10 | 47.8 |
| Control | — | 24.3 |

As can be seen in Table 4, the compounds prepared in the Example increased gastric emptying roughly two-fold, compared to the control, demonstrating that the compounds of the present invention function to improve gastric emptying of normal rats.

### INDUSTRIAL APPLICABILITY

As described hitherto, the novel quinoline derivative compounds, optical isomers thereof, pharmaceutically acceptable salt thereof, and hydrates or solvates thereof according to the present invention activate gastrointestinal motility. Thus, compositions comprising the novel quinoline derivative compounds, optical isomers thereof, pharmaceutically acceptable salt thereof, and hydrates or solvates thereof can be effectively applied to the prophylaxis and therapy of functional dyspepsia or various symptoms of gastrointestinal motility disorders.

1. A quinoline derivative compound represented by the following Chemical Formula 1, an optical isomer thereof, a pharmaceutically acceptable salt thereof, or a hydrate or solvate thereof: 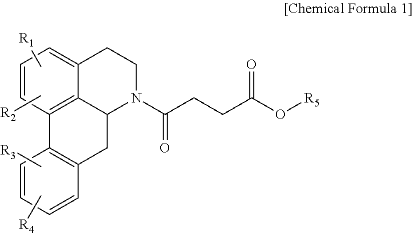
wherein for Chemical Formula 1:
R₁ to R₄ are independently —H or methoxy; and
R₅ is linear or branched alkyl of C₁ to C₄.

2. The quinoline derivative compound of claim 1 represented by Chemical Formula 1, the optical isomer, the pharmaceutically acceptable salt, or the hydrate or solvate thereof, wherein the quinoline derivative compound represented by Chemical Formula 1 is a compound represented by the following Chemical Formula 2: 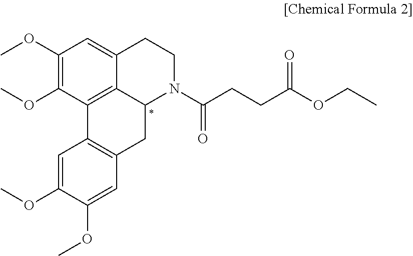
wherein represents an asymmetric carbon atom.

3. The quinoline derivative compound of claim 1 represented by Chemical Formula 1, the optical isomer, the pharmaceutically acceptable salt, or the hydrate or solvate thereof, wherein the quinoline derivative compound represented by Chemical Formula 1 is (S)-4-oxo-4-(1,2,9,10-tetramethoxy-4,5,6a,7-tetrahydro-dibenzo[de,g]quinoline-6-yl)-butyric acid ethyl ester.

4. A method for preparing a compound represented by the following Chemical Formula 1, an optical isomer thereof, a pharmaceutically acceptable salt thereof, and a hydrate or solvate thereof, comprising reacting a compound represented by the following Chemical Formula 3 or an optical isomer thereof with a compound represented by the following Chemical Formula 4 or an optical isomer thereof in presence of a base: 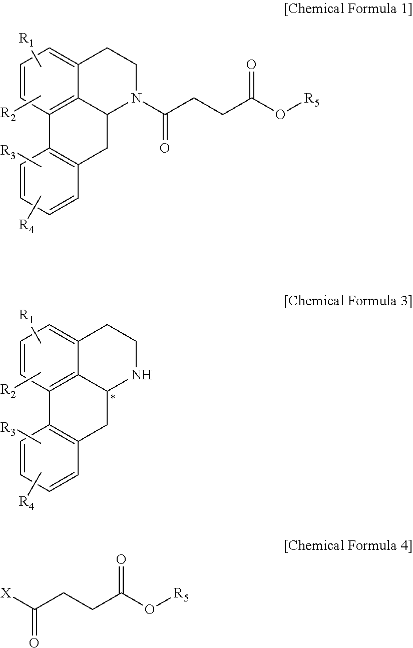
wherein for Chemical Formula 1, 3 and 4:
R₁ to R₄ are independently —H or methoxy;
R₅ is linear or branched alkyl of C₁ to C₄;
X is Cl, Br or I; and
represents an asymmetric carbon atom.

5. The method of claim 4, wherein R₁ to R₄ are methoxy, R₅ is ethyl, and X is Cl.

6. A pharmaceutical composition for prevention or treatment of a gastrointestinal motility disorder, comprising a novel quinoline derivative compound represented by the following Chemical Formula 1, an optical isomer thereof, a pharmaceutically acceptable salt thereof, or a hydrate or solvate thereof: 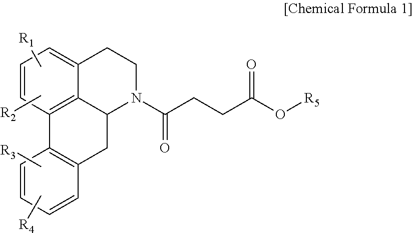
wherein for Chemical Formula 1:
R₁ to R₄ are independently —H or methoxy; and
R₅ is linear or branched alkyl of C₁ to C₄.

7. The pharmaceutical composition of claim 6, wherein the compound represented by Chemical Formula 1 is (S)-4-oxo-4-(1,2,9,10-tetramethoxy-4,5,6a,7-tetrahydro-dibenzo[de,g]-quinoline-6-yl)-butyric acid ethyl ester.
